(19) United States
(12) Patent Application Publication
(10) Pub. No.: US 2024/0261129 A1
(43) Pub. Date: Aug. 8, 2024

(54) SHOE AND METHOD FOR MODIFYING MISALIGNMENT OF A FOOT

(71) Applicant: Eric Ward, Aspen, CO (US)

(72) Inventor: Eric Ward, Aspen, CO (US)

(21) Appl. No.: 18/430,248

(22) Filed: Feb. 1, 2024

Related U.S. Application Data
(60) Provisional application No. 63/483,122, filed on Feb. 3, 2023.

(51) Int. Cl. A61F 5/14; A43B 7/1415; A43B 7/1468

(52) U.S. Cl. CPC A61F 5/14; A43B 7/1415; A43B 7/1468

(57) ABSTRACT

Orthotic shoes and methods for making orthotic shoes and components of the same are disclosed, including a method comprising forming an opening between an upper portion and a lower portion of a base member of a shoe, the opening extending along a side of the intermediate member; positioning an orthotic wedge having a predetermined orthotic angle within the opening of the base member; and securing the orthotic wedge to the upper portion and the lower portion. The method may further include determining or receiving the orthotic angle and forming or selecting the orthotic wedge based on the orthotic angle.

### REFERENCE TO RELATED APPLICATIONS

The present patent application claims priority to the provisional patent application identified by U.S. Ser. No. 63/483,122, filed on Feb. 3, 2023. The entire content of the provisional patent application identified by U.S. Ser. No. 63/483,122 is hereby incorporated herein by reference.

### TECHNICAL FIELD

The present disclosure is directed to a shoe designed to modify and correct misalignment of the knee, ankle, and/or foot of a user. More specifically, the present disclosure relates to a shoe and method of modifying and correcting the misalignment of the knee, ankle, and/or foot of the user.

### BACKGROUND ART

From a simplistic point of view the bipedal motion of walking is akin to the motion of a pendulum. A pendulum is a device that transforms kinetic energy of motion into potential energy, and then back into kinetic energy. As the pendulum moves through the bottom of its arc, the pendulum's velocity and thus its kinetic energy reaches a maximum. Thereafter that kinetic energy is converted back to potential energy as the pendulum comes to rest at its peak. With each step the human body is literally an inverted pendulum. But while a pendulum is incredibly efficient, the human body has some inconsistencies. One such instance is when that pendulum, that is, the foot, is not naturally aligned to the “man-made” flat ground.

An individual standing upright on two feet is a very stable, static structure. The structure that is formed with the legs (two bases of support), hip girdle (the cross beam), and the torso (the third pillar), is a very stable geometric structure. The transverse extension of each foot with respect to the geometric plane formed by the two legs further stabilizes the structure. Indeed, an individual standing still with their legs shoulder width apart represents a very stable and strong static structure.

In such a configuration, the foot, knee, and hip, which are aligned, have a load-bearing axis on a line running down the middle of the leg, through the hip, knee, and ankle. In a static configuration, this line exists in what is referred to as the coronal or frontal plane. The coronal plane divides the body lengthwise, anterior from posterior, such that the face is separated from the back of the head, the chest from the back, the palms from the back of the hands, and the shins from the calves. But when walking, the body performs a pendulum motion about the sagittal plane. The sagittal plane bisects the left and right sides of the body longitudinally. This plane runs down the center of the head and torso, and between the legs and feet.

As an individual walks or runs, the neuro-muscular control system (the proprioceptive system), controls the bones, brain, and muscles in real time to maintain a balanced movement of the center of gravity, by shifting weight from one side of the sagittal plane to the other. In essence, when a person walks (or runs), the person leans on and balances on one of the bases of support of the person, while repositioning the other. However, problems arise from the fact that the ends of each pendulum are not perfect nor symmetric. In fact, each has natural measurable biases relative to flat ground.

In a normal gait, the outside of the heel makes initial contact with the ground as an individual walks or runs. As the center of mass moves forward and the foot becomes fully weighted, the foot rolls inward or pronates because of structural biases as it comes in complete contact with the ground (during midstance) where it can support the entire body weight effectively. The rolling in of the foot optimally distributes the forces of impact. This movement is critical to proper shock absorption and a functional amount of pronation, as defined herein, is required for the foot to function properly, relative to flat ground.

The opposite natural condition of the foot is called supination, which occurs as the outside of the heel makes initial contact with the ground and the normal inward movement of the foot occurs, however, the inward movement is not in the “functional range”. So now instead of the muscles allowing the structural collapse, the muscles need to intervene and control the structural collapse by firing a sustained tension to control the collapse, thus creating an outward movement of the foot which is understood to be “supination”, i.e., a natural muscular bias.

Consequently, forces of impact are concentrated on a smaller area of the foot (the outside part), and are not distributed as efficiently as with functional pronation.

Supination is the opposing range of motion to pronation and refers to the outward roll of the foot during normal bipedal motion. This movement is only possible through muscular intervention.

Pronation and supination, as they are classically understood, attempt to describe a complex simultaneous three-dimensional movement of the skeletal system within the foot and ankle. However, in this understanding there is no explanation as to the complex role or involvement of the neuromuscular system which originates in the foot. Other terms such as inversion vs. eversion, plantar flexion vs. dorsiflexion, and abduction vs. adduction describe various aspects of the complex task of bipedal motion. The dynamic interaction between the skeletal structure of the foot and proprioceptive, neuromuscular counterparts adds complexity.

In simple terms, pronation may be considered to be a natural skeletal bias of the foot. This structural bias, is answered by its opposing muscular bias. Supination is thus the muscular over-reaction or over compensatory bias to a structural collapse. Moreover, while the musculature biases the foot toward supination, the skeletal structure biases the foot to pronate. To efficiently and effectively achieve and maintain bipedal motion, a balance between the cyclical activities of both pronation and supination must occur. In this understanding of the foot dynamics, the more a person structurally pronates, the more the person muscularly supinates.

The harmony of the skeletal and neuro-muscular systems involved in walking and running is impeded when proper foot, ankle, and knee alignment is not maintained. Unlike the very stable structure of an individual standing still with their legs spread, walking and running is inherently unstable, and any misalignment of the mechanical components requires constant muscular compensation every step of the way. The compensation requires energy and reduces overall endurance. Lastly, misalignment can ultimately result in overuse injuries and is the trigger mechanism to many chronic pain symptoms.

There are many orthotic devices and supplemental footbeds designed to modify and correct misalignment of the knee, ankle and foot. These supplemental footbeds are placed in shoes to correct misalignment. However, each person may have misalignments specific to their body. There are currently not any shoes constructed to take into account and correct each person's misalignment. The inventive concepts in the present disclosure solve this and other problems with misalignment.

### BRIEF DESCRIPTION OF DRAWINGS

FIG. 1 is an exemplary, perspective view of a shoe in a first orientation, wherein the base member of the shoe has an opening, in accordance with the present disclosure.

FIG. 2 is a cross-sectional, front view of the exemplary shoe having the opening taken along the line 2-2 as seen in FIG. 1.

FIG. 3 is a side view of the shoe of FIG. 1 in a second orientation in accordance with the present disclosure.

FIG. 4 is an exemplary, perspective view of an orthotic wedge in accordance with the present disclosure.

FIG. 5 is a side view of the shoe of FIG. 1 having an exemplary sole member is accordance with the present disclosure.

FIG. 6 is an exemplary, perspective view of the shoe of FIG. 1 having the orthotic wedge positioned within the opening of the base of the shoe in accordance with the present disclosure.

FIG. 7 is an exemplary, perspective view of the shoe of FIG. 1 having a shaped, orthotic wedge positioned within the opening of the base of the shoe in accordance with the present disclosure.

FIG. 8 is a cross-sectional, front view of the shoe having the shaped, orthotic wedge positioned within the opening of the shoe taken along line 8-8 in FIG. 7.

FIG. 9 is a block diagram of an exemplary method of constructing a shoe configured for modifying the misalignment of a user of the shoe in accordance with the present disclosure.

FIG. 10 is a cross-sectional, front view of an exemplary unitary-base shoe constructed in accordance with the present disclosure.

FIG. 11 is a block diagram of an exemplary method of constructing a unitary-base shoe configured for modifying the misalignment of a user of the shoe in accordance with the present disclosure.

### DETAILED DESCRIPTION

Before explaining at least one embodiment of the presently disclosed and/or claimed inventive concept(s) in detail, it is to be understood that the presently disclosed and/or claimed inventive concept(s) is not limited in its application to the details of construction and the arrangement of the components or steps or methodologies set forth in the following description. The presently disclosed and/or claimed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and/or claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the presently disclosed and/or claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the presently disclosed and/or claimed inventive concept(s) have been described in terms of preferred embodiments, it will be apparent to those skilled in the art that variations may be applied to the articles and/or methods and in the steps or in the sequence of steps of the method described herein without departing from the concept, spirit, and scope of the presently disclosed and/or claimed inventive concept(s).

As utilized in accordance with the present disclosure, the following terms, unless otherwise indicated, shall be understood to have the following meanings.

The use of the word “a” or “an” when used in conjunction with the term “comprising” may mean “one”, but it is also consistent with the meaning of “one or more”, “at least one”, and “one or more than one”. The use of the term “or” is used to mean “and/or” unless explicitly indicated to refer to alternatives only if the alternatives are mutually exclusive.

As used herein, qualifiers “substantially,” “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, inherent variation of error for the quantifying device, the method being employed to determine the value, and combinations thereof, for example.

The use of the term “at least one” or “one or more” will be understood to include one as well as any quantity more than one. In addition, the use of the term “at least one of X, Y, and Z” or “one or more of X, Y, and Z”, will be understood to include X alone, Y alone, and Z alone, as well as any combination of X, Y, and Z.

The use of ordinal number terminology (i.e., “first”, “second”, “third”, “fourth”, etc.) is solely for the purpose of differentiating between two or more items and is not meant to imply any sequence or order or importance to one item over another or any order of addition, for example, unless specifically defined otherwise.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

The presently disclosed and claimed inventive concept(s) relates generally to a shoe and a method of making a shoe that upon wearing by an individual will reduce the misalignment of a foot of an individual. The following disclosure illustrates the exemplary shoe by way of example, but in no way limiting, as a sandal. The shoe described below may be an athletic shoe, a sneaker, a boot, a dress shoe, a flat, a wedge, or the like.

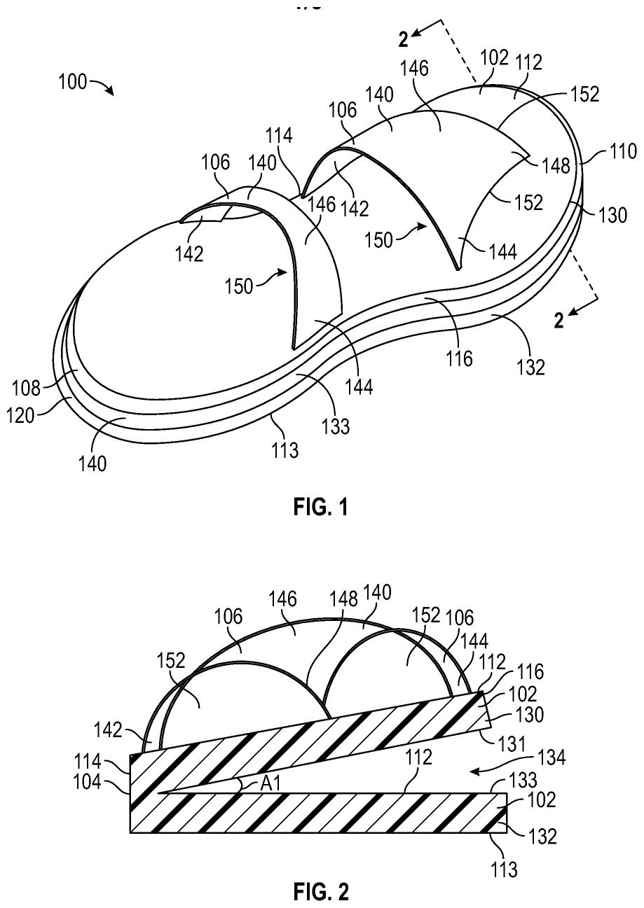

Referring now to the figures, FIG. 1 is a perspective view of an exemplary shoe 100 in accordance with the present disclosure. The shoe 100 may have a base member 102 and at least one upper shoe member 106.

The base member 102 of the shoe 100 may have a first wall 108, a second wall 110 positioned opposite the first wall 108, a top surface 112, a bottom surface 113 positioned opposite the top surface 112, a first side 114, a second side 116 positioned opposite the first side 114.

The base member 102 may also have an intermediate member 120 extending between the first wall 108, the second wall 110, the first side 114, and the second side 116. The intermediate member 120 may have an upper portion 130, and a lower portion 132, positioned opposite the upper portion 130. The upper portion 130 of the intermediate member 120 may have an interior surface 131 positioned opposite the top surface 112 of the base member 102. The lower portion 132 may have an interior surface 133 positioned opposite the bottom surface 113 of the base member 102. The base member 102 may be configured to be in a first orientation, such that the upper portion 130 of the intermediate member 120 may be positioned away from the lower portion 132 of the intermediate member 120 thereby defining an opening 134 (see FIGS. 1 and 2).

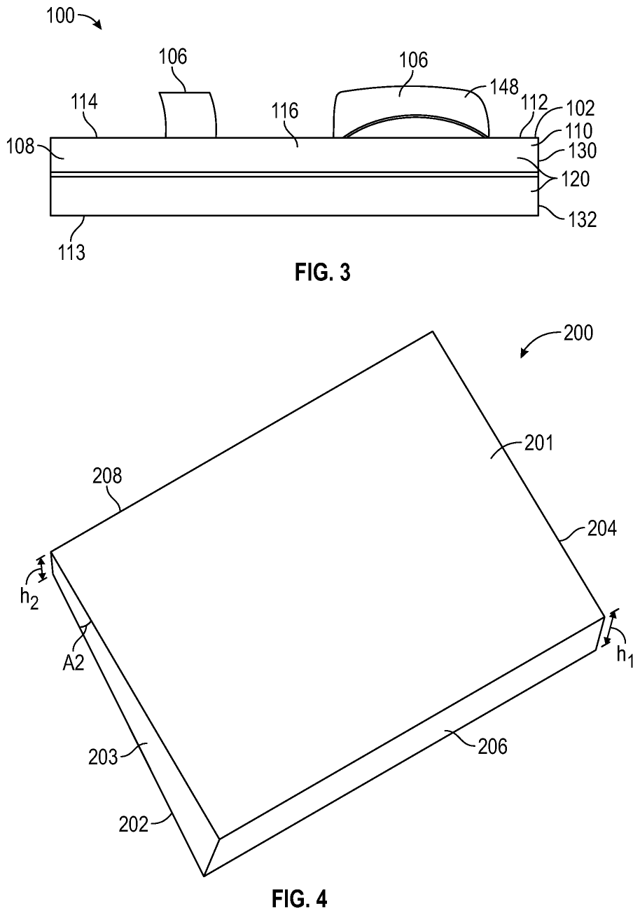

The opening 134 of the base member 102 may be at a first angle A1 (see FIG. 2). The first angle A1 of the opening 134 of the base member 102 may be in a range of 0°-16°. As seen in FIG. 3, the base member 102 may also be configured to be in a second orientation, such that the upper portion 130 of the intermediate member 120 may be positioned adjacent to the lower portion 132 of the intermediate member 120 so that an orthotic angle (as explained below) is effectively zero degrees.

In some implementations, the base member 102 may not have the intermediate member 120, and the upper portion 130 and lower portion 132 may be of the base member 102 rather than of the intermediate member 120.

The base member 102 may comprise rubber, leather, wood, rope, or the like.

Referring now to FIGS. 2 and 8, the hinge 104 of the base member 102 of the shoe 100 may be positioned between and connected to the upper portion 130 and the lower portion 132 of the intermediate member 120 of the base member 102. In some non-limiting embodiments, the hinge 104 may be positioned adjacent to the first side 122 of the base member 102, thereby connecting the upper portion 130 and the lower portion 132 of the intermediate member 120. In other non-limiting embodiments, the hinge 104 may be positioned adjacent the second side 124 of the base member 102, thereby connecting the upper portion 130 and the lower portion 132 of the intermediate member 120. The hinge 104 may be configured to movably position the upper portion 130 of the intermediate member 120 away from the lower portion 132 of the intermediate member 120 thereby defining the opening 134, as seen FIGS. 1-2 and 7-8, thereby positioning the base member 102 of the shoe 100 in the first orientation.

In some non-limiting embodiments, the hinge 104 may be configured to movably position the upper portion 130 of the intermediate member 120 adjacent to the lower portion 132 of the intermediate member 120, thereby positioning the base member 102 of the shoe in the second orientation as seen in FIG. 3.

In some non-limiting embodiments, the hinge 104 may be a portion of the upper portion 130 and the lower portion 132 of the intermediate member 120 of base member 102 (as seen in FIG. 2). In other non-limiting embodiments, the hinge 104 may be separate from the upper portion 130 and the lower portion 132 of the intermediate member 120. By way of example, but in no way limiting, the hinge 104 may be connected to and thereby connect the upper portion 130 and the lower portion 132 of the intermediate member 120.

An example of a hinge 104 that may be separate from the upper portion 130 and the lower portion 132 of the intermediate member 120 may be a movable device. In some non-limiting embodiments, the movable device may be jointed. In some non-limiting embodiments, the movable device may be a mechanical hinge or the like.

As seen in FIGS. 1-3 and 5-8, the upper shoe member 106 of the shoe 100 may have at least one strap 140. In some non-limiting embodiments, the at least one strap 140 of the upper shoe member 106 may have a first end 142, a second end 144, and a middle portion 146. In some non-limiting embodiments, the first end 142 of the at least one strap 140 may be positioned on the top surface 112 of the base member 102, adjacent to the first side 114 of the base member 102 and the second end 144 of the at least one strap 140 may be positioned on the top surface 112 and adjacent to the second side 116 of the base member 102 such that the middle portion 146 of the strap 140 is not connected to the top surface 112 of the base member 102 thereby defining an opening 150. The opening 150 may be configured to receive a portion of the foot of an individual (see FIG. 1).

In some non-limiting embodiments, the strap 140 of the upper shoe member 106 may have a front member 148 extending away from the middle portion 146. The front member 148 may be positioned on the top surface 112 of the base member 102 thereby defining one or more opening 152. The front member 148 may be configured to be positioned between two toes of the foot of the individual, thereby allowing the toes of the foot of the individual to extend away from the one or more opening 152.

In some implementations, the first end 142, the second end 144, and the front member 148 of the at least one strap 140 of the upper shoe member 106 may be connected to the top surface 112 of the base member 102 via a fastener or the like. The fastener may be snap hooks, cord locks, zipper pulls, hook-and-loop fasteners, stitching, or the like. The at least one strap 140 may be leather, a cloth-like material, or the like.

Referring now to FIG. 4, in some non-limiting embodiments, the base member 102 of the shoe 100 may have an orthotic wedge 200. The orthotic wedge 200 may have a top surface 201, a bottom surface 202 positioned opposite the top surface 201, a front wall 203, a back wall 204 positioned opposite the front wall 203, a first sidewall 206 having a first height h1, a second sidewall 208 having a second height h2 positioned opposite the first sidewall 206, wherein the first height h1 of the first sidewall 206 is greater than the second height h2 of the second sidewall 208, thereby defining an orthotic angle A2 of the orthotic wedge 200. The orthotic wedge 200 may be configured to optimize the angle of the foot of the individual relative to the ground to correct supination and/or pronation so that the top surface 112 and the bottom surface 113 are at a non-zero orthotic angle A2.

In some non-limiting embodiments, the orthotic wedge 200 may be constructed of one or more of rubber, plastic, nylon, carbon fiber, metal powders, binding solutions, leather, vegetable materials, or the like.

In some non-limiting embodiments, the orthotic wedge 200 may be constructed via a 3D printer. The 3D printer may be provided with a three-dimensional model of the orthotic wedge 200 and the 3D printer may “print” the orthotic wedge 200 utilizing the three-dimensional model. When a three-dimensional model is used to form the orthotic wedge 200, a predetermined series of computer instructions may be supplied to a 3D printer to construct the orthotic wedge 200. In some implementations, the 3D printer may extrude liquified material, powdered material, wire, and/or sheets, to form two-dimensional cross-sections in layers in order to form the three-dimensional orthotic wedge 200.

In some embodiments, the top surface 201, and the bottom surface 202 of the orthotic wedge 200 are planar surfaces. In some non-limiting embodiments, the top surface 201 of the orthotic wedge 200 may be configured to correspond and be secured to the interior surface 131 of the upper portion 130 of the intermediate member 120. In some non-limiting embodiments, the bottom surface 202 of the orthotic wedge 200 may be configured to correspond and be secured to the interior surface 133 of the lower portion 132 of the intermediate member 120.

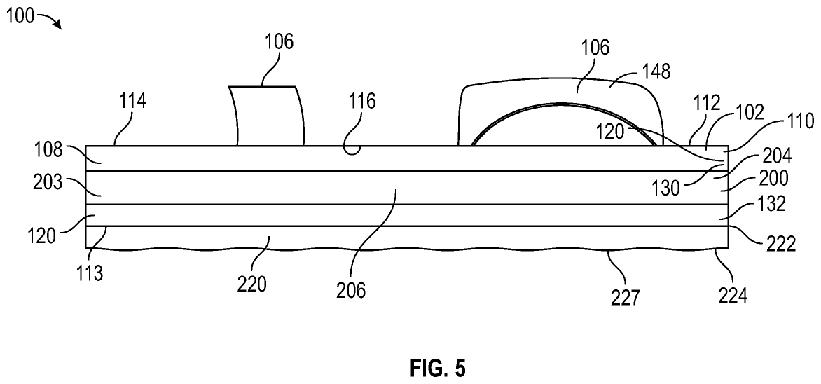

Referring now to FIG. 5, in some non-limiting embodiments, the shoe 100 may also have a sole member 220. The sole member 220 may have a top surface 222, a bottom surface 224 positioned opposite the top surface 222, and a central member 226 extending between the top surface 222 and the bottom surface 224 of the sole member 220. The top surface 222 of the sole member 220 may be positioned adjacent and attached to the bottom surface 113 of the base member 102, thereby connecting the sole member 220 to the bottom surface 113 of the base member 102 of the shoe. The sole member 220 may have one or more traction member 227 formed on the bottom surface 224 of the sole member 220 configured to provide traction, contribute to the rigidity of the shoe 100, and/or contribute to the flexibility of the shoe 100. The material of the sole member 220 of the shoe 100 may comprise one or more of rubber, plastic, or the like.

In some implementations, the shoe 100 may comprise the base member 102 having the first wall 108, the second wall 110, the upper portion 130 extending between the first and second walls 108, 110, and a lower portion 132 positioned opposite the upper portion 130 and hingedly connected to the upper portion 130 so as to form the angled opening having the first angle A1 between the upper portion 130 and the lower portion 132. The orthotic wedge 200 may be positioned within the angled opening formed by the upper portion 130 and the lower portion 132, the orthotic wedge 200 having the top surface 201 and the bottom surface 202 positioned opposite from, and at the orthotic angle A2 to, the top surface 201, wherein the orthotic angle A2 is smaller than the first angle A1 of the angled opening.

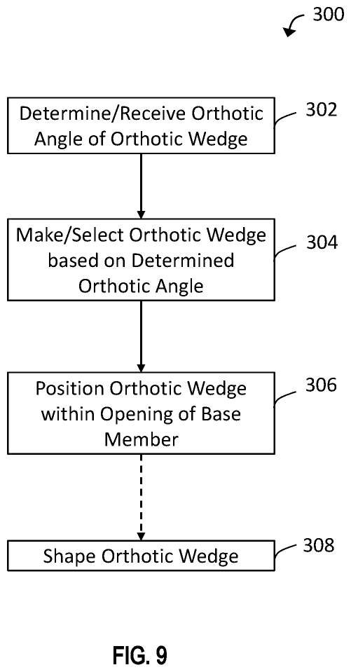

Referring now to FIG. 9, shown therein is a method 300 of creating an exemplary shoe 100 configured for modifying the misalignment of the foot of a user of the shoe 100. According to one embodiment of the present disclosure, in a first step 302, an operator may determine or receive the orthotic angle A2 of the orthotic wedge 200. In some non-limiting embodiments, determining of the orthotic angle A2 may be by performing an angular alignment assessment of the user's foot. The angular alignment assessment of the user's foot may be accomplished in a manner described in U.S. Patent Publication No. 2021/0093229A1, the entirety of which is incorporated herein by reference.

The orthotic angle A2 of the orthotic wedge 200 when applied within the remainder of the shoe 100 may be configured to align a foot, ankle, lower leg, knee, upper leg, and hip of the user of the shoe 100.

The orthotic angle A2 may be between 0.5 degree to 16 degrees. In some embodiments, the orthotic wedges 200 may be pre-manufactured in 0.5 degree increments such that the operator (the person making the shoe 100) has a set of orthotic wedges 200 having various orthotic angles A2. Other increments for the orthotic wedges 200 may also be used.

In a second step 304, once the orthotic angle A2 is known, the orthotic wedge 200 may be made or selected from the set of orthotic wedges 200.

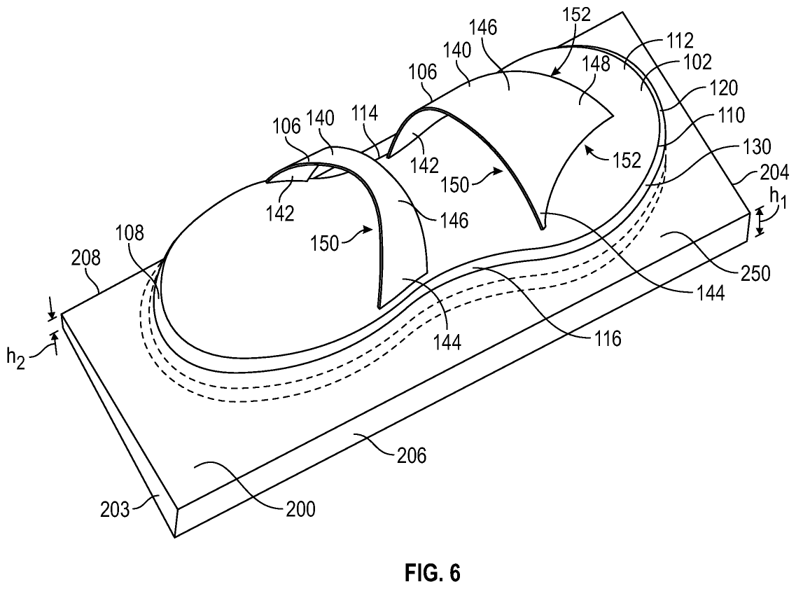

In a third step 306, the base member 102 of the shoe 100 may be positioned in the first orientation, i.e., the upper portion 130 and the lower portion 132 of the intermediate member 120 may be partially separated to form the opening 134, such that the first angle A1 of the opening 134 may be greater than or equal to the orthotic angle A2 of the orthotic wedge 200 so that the orthotic wedge 200 may be positioned within the opening 134 and secured to the upper portion 130 and the lower portion 132. Then, the orthotic wedge 200 may be positioned within the opening 134 of the base member 102 such that the second sidewall 208 of the orthotic wedge 200 may be positioned adjacent the hinge 104 and the first sidewall 206 of the orthotic wedge 200 may extend away from the base member 102 of the shoe 100 (see FIG. 6) thereby defining an excess material 250 (see FIG. 6) of the orthotic wedge 200.

Once the orthotic wedge 200 is positioned within the opening 134, the orthotic wedge 200 may be secured to the upper portion 130 and the lower portion 132 in any suitable manner, such as by bonding the orthotic wedge 200 to the upper portion 130 and the lower portion 132. In other non-limiting embodiments, the top surface 201 of the orthotic wedge 200 may be secured to the upper portion 130 of the intermediate member 120 and the bottom surface 202 of the orthotic wedge 200 may be secured to the lower portion of the orthotic wedge 200. In some non-limiting embodiments, the top surface 201 of the orthotic wedge 200 may be secured to the interior surface 131 of the upper portion 130 of the intermediate member 120. In other non-limiting embodiments, the bottom surface 202 of the orthotic wedge 200 may be secured to the interior surface 133 of the lower portion 132 of the intermediate member 120. In other non-limiting embodiments, the top surface 201 of the orthotic wedge 200 may be secured to the interior surface 131 of the upper portion 130 of the intermediate member 120 and the bottom surface 202 of the orthotic wedge 200 may be secured to the interior surface 133 of the lower portion 132 of the intermediate member 120. The orthotic wedge 200 may be secured to the intermediate member 120 via one or more mechanical member, an adhesive substance, or the like. The mechanical member may be a fastener, such as a button or the like. The adhesive substance may be a glue, rubber cement, or the like.

In some non-limiting embodiments, the method 300 may include forming the upper portion 130 and the lower portion 132 of the intermediate member 120, the hinge 104, and the opening 134 of the intermediate member 120 of the base member 102 of the shoe 100 via slicing through a portion of the intermediate member 120.

In some non-limiting embodiments, slicing through a portion of the intermediate member 120 of the base member 102 may be from the first side 114 of the base member 102 in the direction of the second side 116 of the base member 102 thereby forming the upper portion 130 of the intermediate member 120, the lower portion 132 of the intermediate member 120, the hinge 104, and the opening 134, such that the opening 134 may extend along the first side 114 of the intermediate member 120. In other non-limiting embodiments, slicing through a portion of the intermediate member 120 of the base member 102 may be from the second side 116 of the base member 102 in the direction of the first side 114 of the base member 102 thereby forming the upper portion 130 of the intermediate member 120, the lower portion 132 of the intermediate member 120, the hinge 104, and the opening 134, such that the opening 134 may extend along the second side 116 of the intermediate member. In some non-limiting embodiments, slicing through a portion of the intermediate member 120 may be via a blade, a knife, a box cutter, a laser cutter, a water cutter, or the like.

In some non-limiting embodiments, the method 300 may include positioning the top surface 222 of the sole member 220 adjacent to the bottom surface 113 of the base member 102 of the shoe 100 and attaching the top surface 222 of the sole member 220 to the bottom surface 113 of the base member 102 of the shoe 100. The sole member 220 may be attached via bonding or a mechanical member. The bonding may be an adhesive substance. The adhesive substance may be a glue, rubber cement, or the like. The mechanical member may be a fastener, such as a staple, stitching or the like. Before or after the sole member 220 is attached to the bottom surface 113 of the base member 102, the upper shoe member 106 is connected to the top surface 112 of the base member 102 via a fastener or the like. The fastener may be snap hooks, cord locks, zipper pulls, hook-and-loop fasteners, stitching, or the like.

In some non-limiting embodiments, the second sidewall 208 of the orthotic wedge 200 may be positioned adjacent to hinge 104 of the shoe 100. As discussed above, the hinge 104 may be on the first side 114 or the second side 116 of the base member 102.

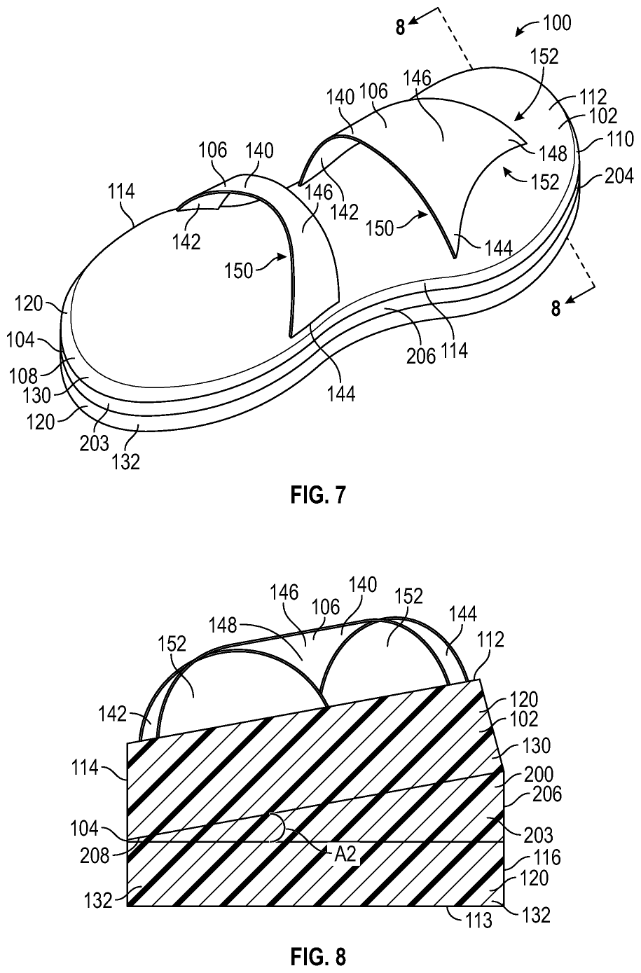

In some implementations, in a fourth step 308, the orthotic wedge 200 may be shaped such that the orthotic wedge 200 extends between the first wall 108, the second wall 110, the first side 114, and the second side 116 of the base member 102; that is, the orthotic wedge 200 extends to a perimeter of the intermediate member 120 of the base member 102 of the shoe 100 (see FIGS. 7 and 8). The orthotic wedge 200 may be shaped by removing the excess material 250 of the orthotic wedge 200 that extends beyond the shoe 100. The orthotic wedge 200 may be shaped via a trimming member, for example. The trimming member may be scissors, razor blade, box cutter, laser cutter, water cutter, or the like. In some implementations the fourth step 908 is not needed, as the orthotic wedge 200 may be initially formed in the shape of the base member 102 of the shoe 100 or to otherwise fit within the shoe 100.

In some implementations, the shoe 100 may be constructed utilizing the base member 102 having the orthotic wedge 200 and utilizing the sole member 220 and/or other components of the shoe 100. However, in some implementations, the shoe construction may occur as part of a separate process.

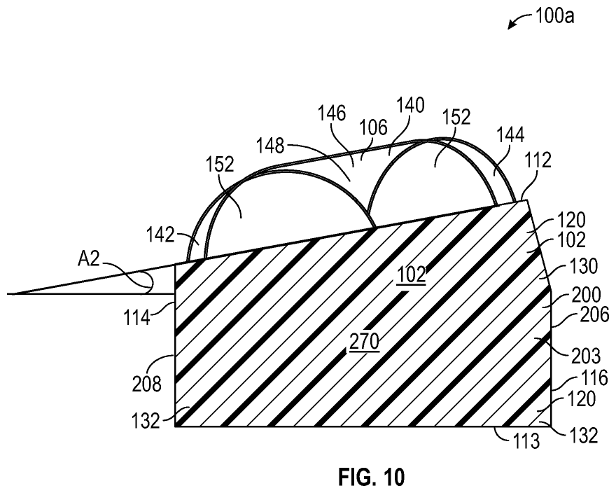

FIG. 10 illustrates an implementation of an exemplary unitary-base shoe 100a constructed in accordance with the inventive concepts disclosed herein. The unitary-base shoe 100a is similar in use and construction to the shoe 100 except as described herein. In the unitary-base shoe 100a, the base member 102 and the orthotic wedge 200, and optionally the sole member 220, may be integrally formed to be a unitary base structure 270. In these embodiments, one or more of, including but not limited to: (a) at least one of the upper portion 130 and the lower portion 132 of the base member 102, (b) the orthotic wedge 200, and (c) optionally, the sole member 220, may be formed together, without the intermediate member 120, the orthotic wedge 200, and the sole member 220 being separately constructed and connected together. In these non-limiting embodiments, the intermediate member 120, the orthotic wedge 200, and the sole member 220 may be constructed together using any suitable process, such as molding and/or a three-dimensional printing process.

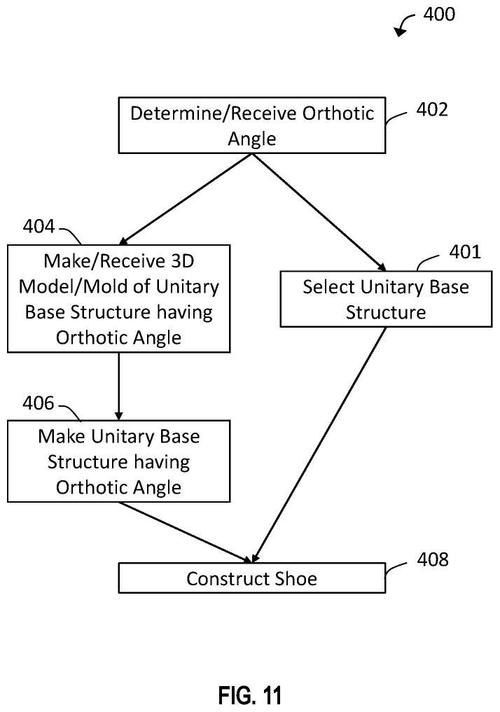

Turning now to FIG. 11, shown therein is a method 400 of creating an exemplary unitary-base shoe 100a configured for modifying the misalignment of the foot of a user of the unitary-base shoe 100a. In a first step 402, the orthotic angle of the unitary base structure 270 may be received or may be determined by performing an angular alignment assessment of the user's foot, as described above.

Determining the orthotic angle A2 for the unitary base structure 270 may be by performing an angular alignment assessment of the user's foot. The angular alignment assessment of the user's foot may be accomplished in a manner described in U.S. Patent Publication No. 2021/0093229A1, the entirety of which is incorporated herein by reference.

The orthotic angle A2 of the unitary base structure 270 (and/or the base member 102) may be configured to align a foot, ankle, lower leg, knee, upper leg, and hip of the user of the unitary-base shoe 100a when in use. The orthotic angle A2 may be between 0.5 degree to 16 degrees.

In some embodiments, a plurality and/or set of unitary base structures 270 (e.g., the combined base member 102 and orthotic wedge 200, and/or the sole member 220) may be pre-manufactured having orthotic angles A2 in 0.5 degree increments such that the person making the unitary-base shoe 100a has a set of unitary base structures 270 having various sizes and orthotic angles A2. Other increments for the orthotic angles A2 of the set of unitary base structures 270 may also be used. Once the orthotic angle A2 is known for a particular foot, then in a second step 401, the unitary base structure 270 having the closest orthotic angle A2 may be made or selected from the plurality of pre-manufactured unitary base structure 270.

In some implementations, in an alternative second step 404, such as when the unitary base structure 270 is being made for a particular user, once the angular alignment assessment of the user's foot is known, then a three-dimensional mold or model for the unitary base structure 270 (including one or more of the base member 102 with intermediate member 120, the orthotic wedge 200, and optionally the sole member 220) may be constructed.

In addition to the orthotic angle A2, the three-dimensional mold or model may also include a particular size of the unitary-base shoe 100a to fit the user's foot and configured for whether the unitary-base shoe 100a is intended to be worn on the user's left foot or right foot.

In a third step 406, in one implementation, once the mold is formed, then rubber, plastic, and/or other material may be added to the mold to form the unitary base structure 270 (such as the combined base member 102, the orthotic wedge 200, and/or the sole member 220). Additionally, or alternatively, when a three-dimensional model is used to form the unitary base structure 270, a predetermined series of computer instructions may be supplied to a 3D printer to construct all or part of the unitary base structure.

The 3D printer may be provided with one or more three-dimensional model of the unitary base structure 270 and the 3D printer may “print” the unitary base structure 270 utilizing the three-dimensional model(s). In some implementations, the 3D printer may extrude liquified material, powdered material, wire, and/or sheets, to form two-dimensional cross-sections in layers in order to form the three-dimensional unitary base structure 270.

When the orthotic wedge 200 is constructed as part of the unitary base structure 270, in some implementations, it may be unnecessary to shape (such as trim) the orthotic wedge 200.

When the sole member 220 is constructed with the unitary base structure 270, then there is no need to separately attach the sole member 220. When the unitary base structure 270 is constructed without the sole member 220, the unitary base structure 270 may be connected to the sole member 220 (including positioning the top surface 222 of the sole member 220 adjacent to the bottom surface 113 of the base member 102 of the shoe 100 and attaching the top surface 222 of the sole member 220 to the bottom surface 113 of the base member 102 of the shoe 100). The sole member 220 may be attached via bonding or a mechanical member. The bonding may be an adhesive substance. The adhesive substance may be a glue, rubber cement, or the like. The mechanical member may be a fastener, such as a staple, stitching or the like. Before or after the sole member 220 is attached to the bottom surface 113 of the base member 102, the upper shoe member 106 is connected to the top surface 112 of the base member 102 via a fastener or the like. The fastener may be snap hooks, cord locks, zipper pulls, hook-and-loop fasteners, stitching, or the like.

In a fourth step 408, the unitary-base shoe 100a may be constructed utilizing the unitary base structure 270. However, in some implementations, the entire unitary-base shoe 100a may be constructed as part of the 3D modeling, molding, and printing steps. Further, in some implementations, the final construction of the unitary-base shoe 100a may occur as part of a separate process.

### Conclusion

In summary, the shoe 100 and the unitary-base shoe 100a may be provided with any orthotic angle A2, which is determined and configured to modify and correct misalignment of the knee, ankle, and foot of a user or a group of users having a similar orthotic angle A2 when using the shoe 100 with the orthotic wedge 200 or the unitary-base shoe 100a.

The elements of the shoe 100 prior to introduction of the orthotic wedge 200 can be configured to have a neutral orthotic angle A2. By applying the orthotic wedge 200 having a desired orthotic angle A2 into the opening 134 and securing the orthotic wedge 200 within the opening 134, or making the unitary base structure 270 to include the orthotic angle A2 as discussed above, a custom shoe designed to correct misalignment for a particular user or group of users is provided.

From the above description, it is clear that the inventive concepts disclosed herein are well adapted to carry out the objects and to attain the advantages mentioned herein as well as those inherent in the inventive concepts disclosed herein. While presently preferred embodiments of the inventive concepts disclosed herein have been described for purposes of this disclosure, it will be understood that numerous changes may be made which will readily suggest themselves to those skilled in the art and which are accomplished within the scope and coverage of the inventive concepts disclosed and claimed herein.

What is claimed is:

1. A shoe, comprising:
a base member having a first sidewall, a second sidewall, a top surface, a bottom surface positioned opposite the top surface, and an intermediate member extending between the first sidewall, the second sidewall, the top surface and the bottom surface, wherein the intermediate member has a first side, a second side positioned opposite the first side, an upper portion, a lower portion, positioned opposite the upper portion, and a hinge connected to the upper portion and the lower portion of the intermediate member such that a portion of the upper portion and the lower portion may be hingedly separated thereby defining an opening; and
at least one upper shoe member connected to the top surface of the base member of the shoe.

2. The shoe of claim 1, wherein the hinge comprises a portion of the upper portion and a portion of the lower portion of the intermediate member.

3. The shoe of claim 1, further comprising a sole member attached to the bottom surface of the base member.

4. The shoe of claim 3, wherein the sole member further comprises a top surface, a bottom surface, positioned opposite the top surface of the sole member, and a central member extending between the top surface of the sole member and the bottom surface of the sole member.

5. The shoe of claim 4, wherein the bottom surface of the sole member has one or more traction member.

6. The shoe of claim 1, wherein the opening extends along the first side of the intermediate member of the base member.

7. The shoe of claim 1, wherein the opening extends along the second side of the intermediate member of the base member.

8. The shoe of claim 1, wherein the base member further comprises an orthotic wedge having an orthotic angle, the orthotic wedge positioned within the opening formed by the upper portion and the lower portion.

9. The shoe of claim 8, wherein the orthotic wedge has a top surface, a bottom surface positioned opposite the top surface of the orthotic wedge, a front wall, a back wall positioned opposite the front wall, a first sidewall having a first height, a second sidewall having a second height positioned opposite the first sidewall, wherein the first height of the first sidewall is greater than the second height of the second sidewall thereby defining an orthotic angle of the orthotic wedge.

10. The shoe of claim 8, wherein the upper portion and the lower portion are positioned at an angle greater than or equal to the orthotic angle of the orthotic wedge.

11. A method, comprising:
forming an opening between an upper portion and a lower portion of an intermediate member of a base member of a shoe, the opening extending along a side of the intermediate member;
positioning an orthotic wedge having an orthotic angle within the opening of the base member;
securing the orthotic wedge to the upper portion and the lower portion; and
shaping the orthotic wedge by removing excess material extending beyond a perimeter of the intermediate member.

12. The method of claim 11, further comprising determining an angle of the orthotic wedge, and selecting the orthotic wedge from a plurality of orthotic wedges having differing orthotic angles.

13. The method of claim 11, further comprising connecting a hinge to the upper portion and the lower portion.

14. The method of claim 11, further comprising slicing through a portion of the intermediate member to the upper portion, the lower portion, and a hinge connecting the upper portion to the lower portion.

15. The method of claim 11, further comprising attaching a sole member to a bottom surface of the base member.

16. The method of claim 15, wherein the sole member of the shoe further comprises a top surface, and attaching the top surface of the sole member to a bottom surface of the base member.

17. The method of claim 16, further comprising forming one or more traction member on a bottom surface of the sole member.

18. The method of claim 11, wherein forming the opening between the upper portion and the lower portion of the intermediate member is defined further as moving at least one of the upper portion and the lower portion to form an angle between the upper portion and the lower portion greater than or equal to the orthotic angle of the orthotic wedge.

19. A shoe, comprising:
a base member having a first sidewall, a second sidewall, a top surface, a bottom surface positioned opposite the top surface, an intermediate member extending between the first sidewall, the second sidewall, the top surface and the bottom surface, wherein the intermediate member has a first side, a second side positioned opposite the first side, at least one of an upper portion and a lower portion, an orthotic wedge having an orthotic angle between 0.5 degrees and 16 degrees being integrally formed with the at least one of the upper portion and the lower portion; and
at least one upper shoe member connected to the base member of the shoe.

20. The shoe of claim 19, wherein the base member is formed via a three-dimensional printing process in which the intermediate member and the orthotic wedge are formed as a unitary structure.

21. A shoe, comprising:
a base member having a first wall, a second wall, an upper portion extending between the first wall and the second wall, and a lower portion positioned opposite the upper portion and hingedly connected to the upper portion so as to form an angled opening between the upper portion and the lower portion, the angled opening positioned at a first angle;
an orthotic wedge positioned within the angled opening formed by the upper portion and the lower portion, the orthotic wedge having a top surface and a bottom surface positioned opposite from, and at an orthotic angle to, the top surface, wherein the orthotic angle is smaller than the first angle of the angled opening; and
at least one upper shoe member connected to the top surface of the base member.

22. The shoe of claim 1, further comprising a sole member attached to the bottom surface of the base member.

23. The shoe of claim 1, wherein the angled opening extends along the first sidewall of the base member.
